(19) United States
(12) Patent Application Publication
(10) Pub. No.: US 2011/0004088 A1
(43) Pub. Date: Jan. 6, 2011

(54) THE ECG SHIRT

(76) Inventor: Kurt Paul Grossman, Newport Beach, CA (US)

Correspondence Address: KURT PAUL GROSSMAN, 605 MAR VISTA DRIVE, NEWPORT BEACH, CA, 92660, US

(21) Appl. No.: 12/464,878

(22) Filed: May 13, 2009

(51) Int. Cl. A61B 5/0408

(52) U.S. Cl. 600/382

(57) ABSTRACT

The electrocardiograph (ECG) Shirt is an extremely elastic non-electric and non-mechanical method that creates a more secure bond to the skin and more accurate and standardized ECG electrode lead placement for men, women, and children. The ECG Shirt reduces unexpected movement or dislocation. Contrasted with much prior art that analyzes errors after the fact the ECG Shirt eliminates the potential for errors to occur. The ECG Shirt is capable of using most ECG devices with different types of electrode lead connectors or pads. The ECG Shirt facilitates not only twelve lead resting ECG testing but also exercise testing, monitoring, fifteen lead, and two different types of eighteen lead ECG testing. The ECG Shirt is a tool for teaching expert ECG electrode lead placement.

### CROSS-REFERENCE TO RELATED APPLICATIONS

N/A—Not Applicable

### STATEMENT REGARDING FEDERALLY SPONSORED RESEARCH OR DEVELOPMENT

N/A—Not Applicable

### REFERENCE TO SEQUENCE LISTING, A TABLE, OR A COMPUTER PROGRAM LISTING COMPACT DISK APPENDIX

N/A—Not Applicable

### BACKGROUND OF THE INVENTION

The invention relates to measuring a physiological characteristic of a patient, and particularly, to an electrocardiograph including a method and apparatus for identifying, specifying, and fixing the relative position of the electrodes connected to the patient.

It is commonly known that ten electrodes and ten leadwires are needed to record and present what is commonly referred to as a twelve lead electrocardiogram (ECG), i.e., a group of twelve signals representing twelve different “views” of the electrical activity in the patient's heart. For standard or resting ECG electrode placement, one electrode is attached to each of the four body limbs at the right wrist, left wrist, right ankle, and left ankle Additionally, six electrodes are attached to the chest over the heart. The ten electrodes connect via several resistor networks to enough amplifiers to record twelve channels of ECG. The twelve leads (i.e., signals) are generally split into two groups comprising the frontal plane and the horizontal plane. The frontal plane leads (I, II, III, aVr, aVl, aVf) are variously referred to as limb leads, Einthoven leads, or bipolar leads. The horizontal plane leads (v1, v2, v3, v4, v5, v6) are likewise variously referred to as precordial leads, chest leads, or unipolar leads.

It is not as commonly known that many issues relating to cardiovascular anomalies are not as easily detected with a twelve lead ECG. There are at least 11 different varieties of myocardial injury, three of which require the use of a fifteen lead and two the use of an eighteen lead ECG for accurate diagnosis. Once the twelve lead has been recorded it is easy to collect the fifteen or eighteen lead ECG and takes about one minute to switch the leads after the twelve lead ECG test is recorded. There are fifteen lead and eighteen lead systems available also.

Clinicians, technicians, and allied healthcare workers vary greatly in each individual placement of the ECG leads and different clinicians may record ECG data that is placed in a patient file. The variety of placement has caused researchers to study the effects. They have concluded that the variation causes significant concern. One specific example of a problem among clinical experts is the wide variation of the correct location for electrode placement for women, particularly women who have large breast size.

Accurate placement of the electrodes on the patient's body surface is required to record a useful ECG using an electrocardiograph or patient monitor. The ideal placement of electrodes for a standard ECG is well defined and accepted within the medical industry. However, routine correct placement of the electrodes in the clinical environment is difficult to achieve for several reasons. First, nurses and ECG technicians are frequently not adequately trained or are too inexperienced to accurately locate the attachment points. Moreover, individual physical characteristics vary widely from patient-to-patient. These variations lead to misinterpretation of the “anatomical guideposts” used to locate the proper attachment points. Additionally, patients sometimes have wounds or bandages that preclude access to the patient's body surface at the proper attachment points. Also, attachment of the electrodes to an ECG machine is often accomplished using long individual ECG leadwires. Even if the electrodes are accurately placed on the patient, the leadwires connecting them to the electrocardiograph may be crossed such that signals are switched at the instrument.

Many inventors in the past have attempted to solve the problem of electrode connection to the chest. Numerous belts, pads, vests, harnesses and strip electrodes have been developed that place a multitude of electrodes into an ordered arrangement to facilitate the attachment of the leads to the patient and eliminate the possibility of some types of attachment errors. In general, these inventions attempt to fix the six horizontal electrodes in relation to each other while adapting to different patient sizes. None of these teachings address the issue of placement of the limb electrodes.

Many attempts have been made to CHANGE how healthcare collects and ECG. The devices include straps, harnesses, buttons, clips, and require as much time as the preparation of the ECG test.

In U.S. Pat. No. 4,608,987 the BACKGROUND OF THE INVENTION states, “Prior art garments typically utilize an elastic cloth front portion specifically tailored to the individual patient. U.S. Pat. No. 2,685,881 teaches a rubber strap for holding a single electrode. Single electrodes, however, are simply not capable of generating a desired complete ECG printout necessary for the cardiologist. U.S. Pat. No. 3,409,007 teaches a vest-like garment fabricated from elastic cloth, with the desired plurality of electrodes. The front portion of the panel preferably has a substantial measure of elasticity in at least two orthogonal dimensions, and band-like appendages are provided for the limb electrodes. U.S. Pat. No. 3,534,727 teaches a garment with flexible elastic electrodes, with the electrodes held in contact with the patient's body by the skin tight garment. U.S. Pat. No. 3,525,330 describes an elastic cloth garment with a grid-like pattern for prescribing the location of electrodes. U.S. Pat. Nos. 4,121,575 and 4,202,344 teach an expandable or stretchable non-conductive strip, such as rubber sheeting, for the six precordial electrodes, while the limb lead electrodes may be of the conventional type. As stated for example in U.S. Pat. No. 4,202,344, the front garment material is stretchable so that when the garment is worn by the patient, the electrodes automatically assume the correct anatomic location on the chest, and thus the position of an electrode moves relative to another electrode depending on the degree the support material is stretched. U.S. Pat. No. 3,409,007 indicates that a portion of the front panel may be provided with multiple thickness of elastic cloth to provide “additional resilient stiffness” to that portion of the garment.”

A very common error for trained and untrained alike is the reversal of the right leg and the left leg and the reversal of the right arm and left arm electrode leads. It is unknown why this occurs so often but some attribute the frequency to the abstract nature of the patient and the healthcare provider facing the patient. When a healthcare provider faces a patient the patient right arm is actually closest to the left arm of the patient.

In some ECG applications the patient is uncooperative or in transport. A certain portion of time ECG tests are sent to hospital settings where a mild sedative is given to insure that the child remains compliant and still during the ECG test.

Children, psychotic patients in institutions, patients during ambulance transport are moving creating the chance for deviations in the ECG record sometimes called a Baseline Artifact.

In other ECG applications the patient must be free to move. Thus, it becomes inconvenient or impossible to place the electrodes on the wrists and ankles Applications where the patient must be free to move include long term recordings, known as Holter; ambulatory patient monitoring, such as telemetry monitoring; and exercise testing on treadmills or bicycles, known as stress testing. In these tests, the wrist and ankle electrode positions are unacceptable for electrode placement due to inconvenience, increased danger of tangling of the lead wires, and increased noise from limbs in motion. Generally, in each of these ECG applications the limb electrodes are moved onto the torso and placed near the shoulders and hips. The Mason-Likar system is one variation of electrode placement on the torso. Twelve-lead bedside monitoring also requires placement of the electrodes on the torso. In each of the systems for alternative electrode placement, useful ECG data is obtained, but the data differs significantly from standard EGC data. Important differences in amplitudes and waveforms occur between standard ECGs and alternative electrode placement ECGs.

### BRIEF SUMMARY OF THE INVENTION

The ECG Shirt takes less time for experienced clinicians, technicians, or novices to place ECG electrode leads on the skin for twelve, fifteen, eighteen, and Mason Likar System lead placement systems. The ECG Shirt applies extremely simple flexible technology that insures a very wide variation in body sizes will not compromise the ECG lead placement. The ECG Shirt uses elastic material used most often in water sports or ballet because of its strength and elasticity.

Additional lightweight strong straps secure the lead wires, electrodes, electrode clips, and electrode pads to employ a three-part connection including the skin and the ECG Shirt. This three-part connection make a much stronger bond to promote the best possible conductivity; reduce movement; and eliminate disconnection.

The ECG Shirt creates de facto standardization of the ECG electrode lead placement that has many clinicians concerned.

Women with large breast size can be accommodated more easily and quickly with the ECG Shirt. The ECG Shirt creates a form of standardization that addresses the concern among clinical experts for the wide variation in the identification of the correct location for electrode placement, particularly in the lateral leads and in women. This has significant implications when comparing ECG in which electrodes have been placed by different clinicians.

The ECG Shirt uses bold visual cues with labels, colors, and numbers in accordance with American and International standards for ECG lead placement so that there will be stark contrast any time an ECG lead is misplaced. There are child, male, and female sizes for people to eliminate the need for shaving hair to stick the electrode leads to the skin.

The design is lightweight mobile durable and flexible and includes the ability to use a standard ECG setup for monitoring. The lead wires are secured in place easily to allow for a full range of motion. Treadmill testing and twelve lead monitoring can be done with the standard twelve lead placement as well as the Mason Likar System or other systems that place the arm and leg leads on the torso.

Because of the flexibility whether the ECG test is done in a clinic or over an extended time for monitoring the leads have little chance of being disconnected or misplaced with motion accidentally.

For the use of pediatric ECG tests there is the added advantage that a parent can be given an ECG Shirt and put it on and wait for the child to fall asleep before taking the test.

The most unique and beneficial feature of the ECG Shirt is the ease of use! In one to two minutes a complete novice with no training can easily place the ECG leads correctly on a person and they will not fall off.

### DESCRIPTION OF THE DRAWINGS

FIG. 1. ECG SHIRT ANTERIOR—FRONT VIEW describes the location of the ECG Electrode Lead Apertures created where healthcare providers will access the patient skin for ECG lead placement looking at the patient chest.

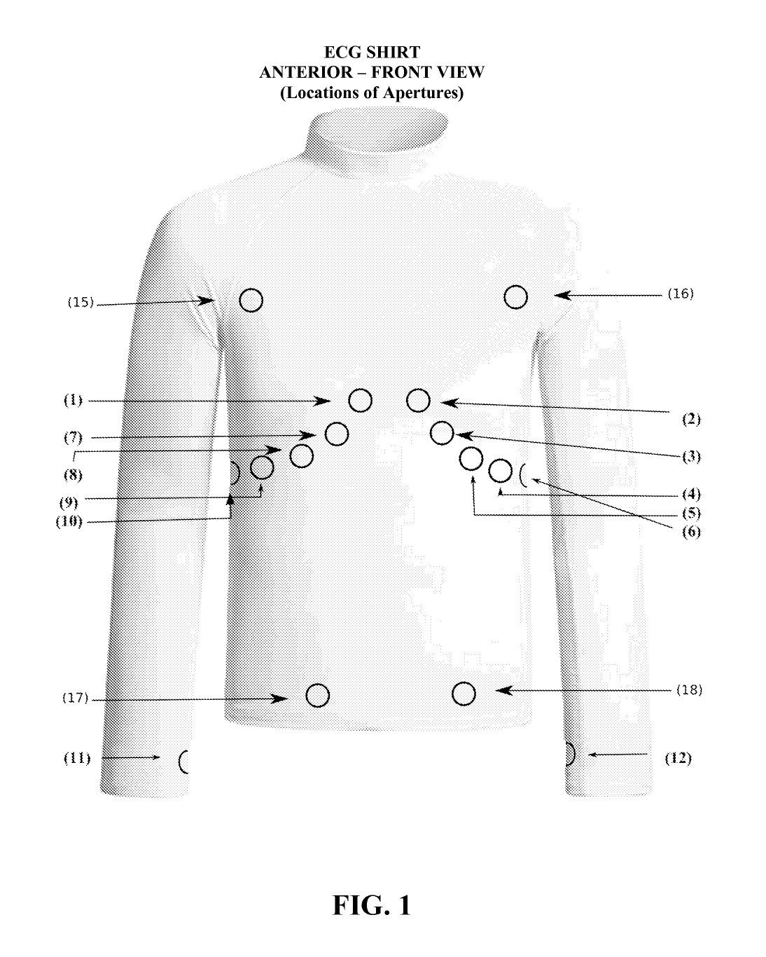

FIG. 2. ECG SHIRT ANTERIOR—FRONT VIEW describes the location of the ECG Electrode Lead Apertures created where healthcare providers will access the patient skin for ECG lead placement looking at the patient legs.

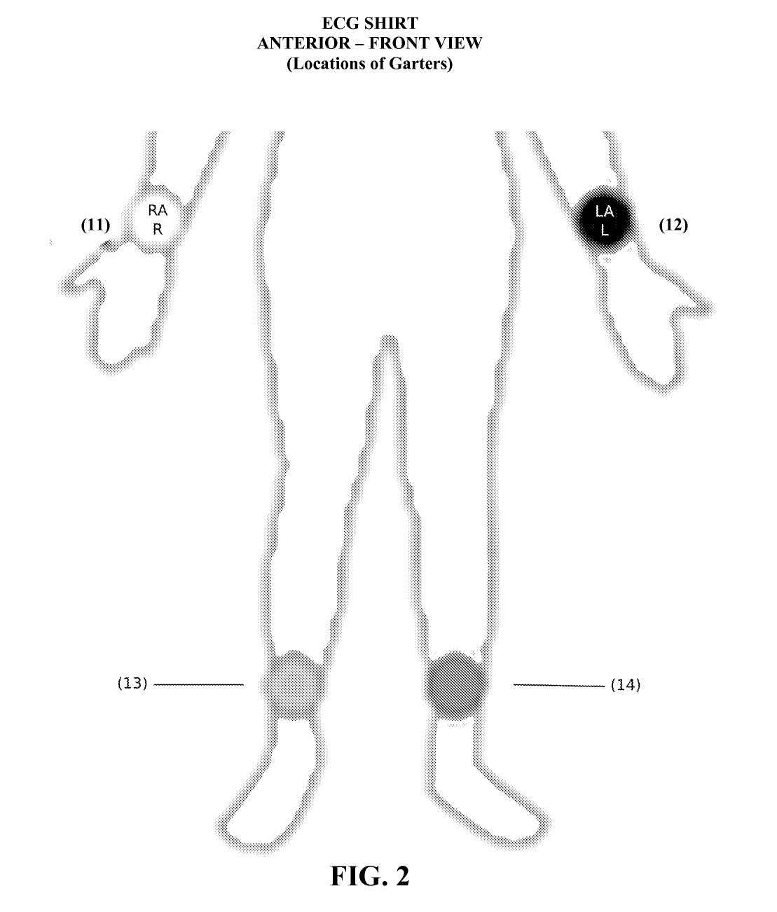

FIG. 3. POSTERIOR or BACK VIEW describes the location of the ECG Electrode Lead Apertures created where healthcare providers will access the patient skin and also illustrates the corresponding anatomical “LINES” (as an additional reference point) for ECG lead placement looking at the patient back.

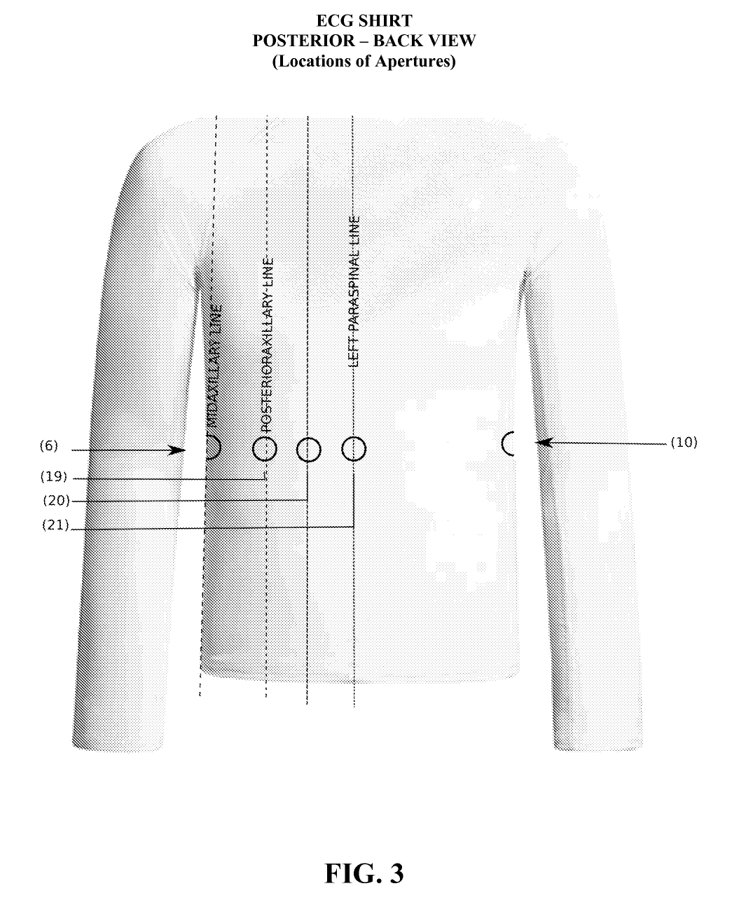

FIG. 4. ANTERIOR or FRONT VIEW describes the location of the harnesses created as guides to contain the ECG lead wires and the fasten the ECG lead wires and ECG electrode clips to the ECG Shirt.

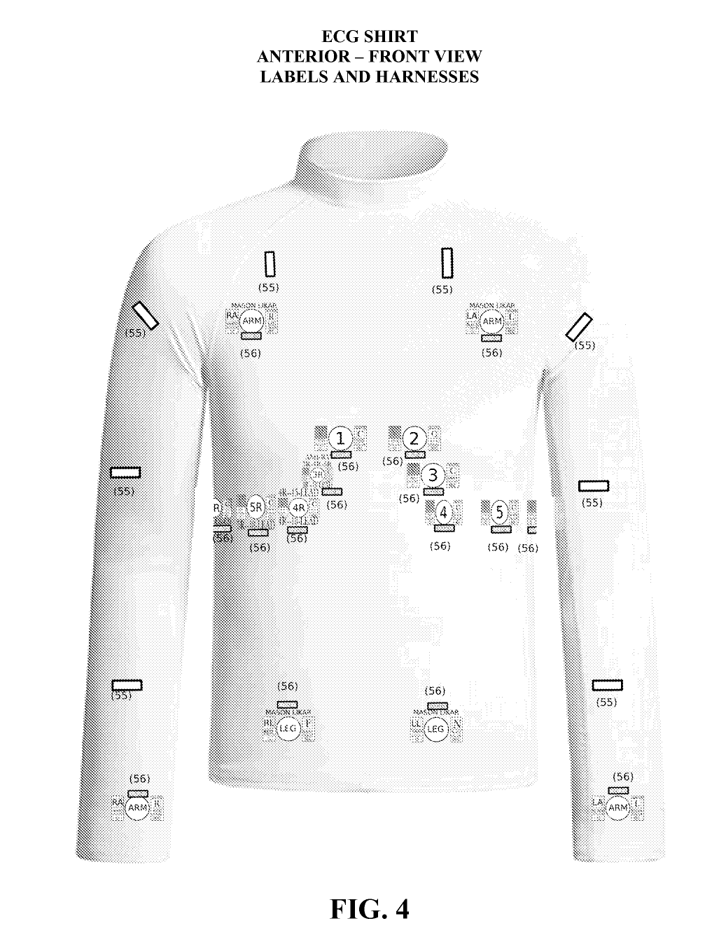

FIG. 5. TOP VIEW describes the elastic band garters that identify the aperture location; stretch out of the way of the aperture; and apply steady pressure on the ECG electrode or ECG electrode pad against the skin.

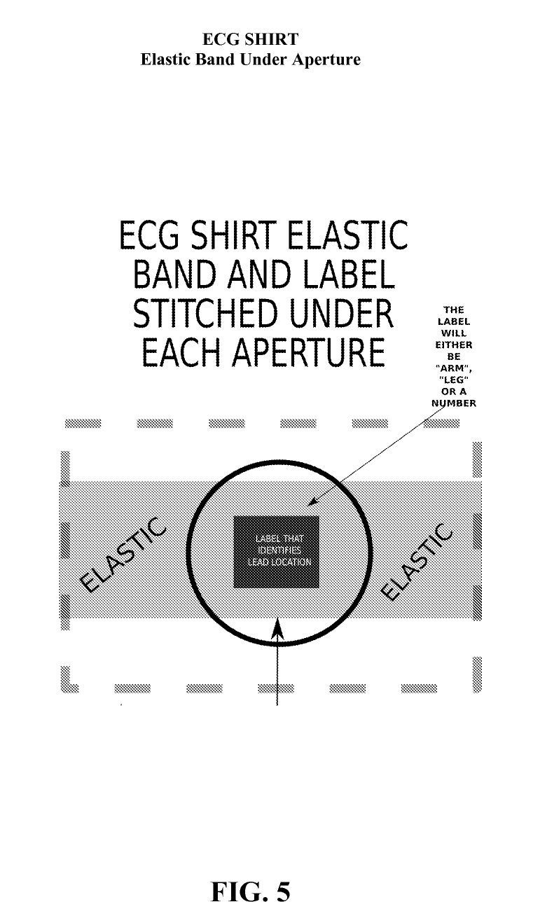

FIG. 6. ECG Shirt Vest

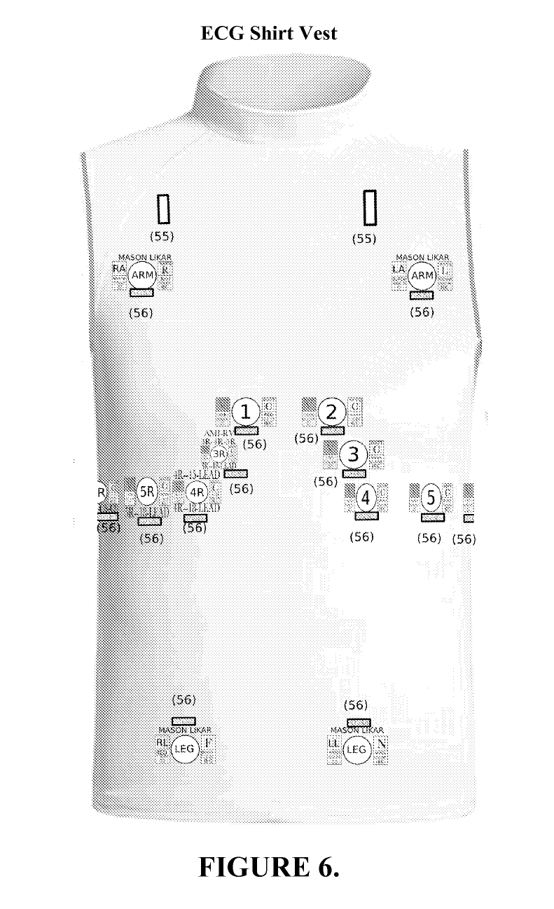

FIG. 7. ECG Shirt Labels

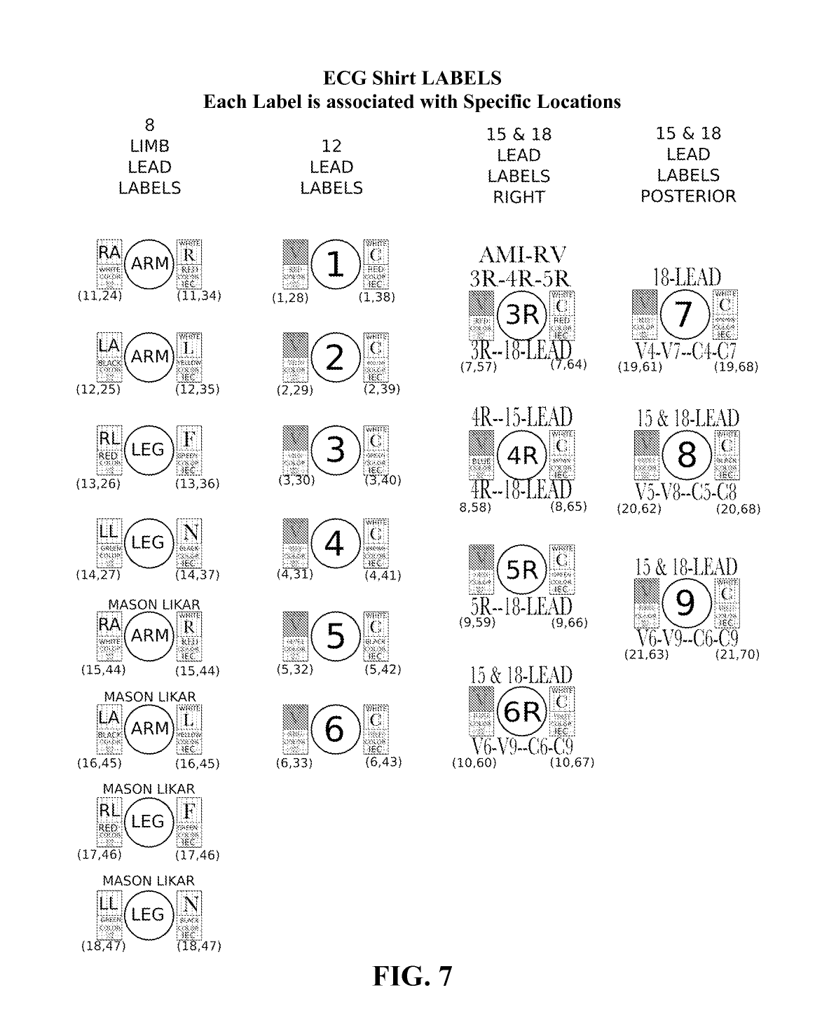

FIG. 8. ECG Shirt Tables 1-3

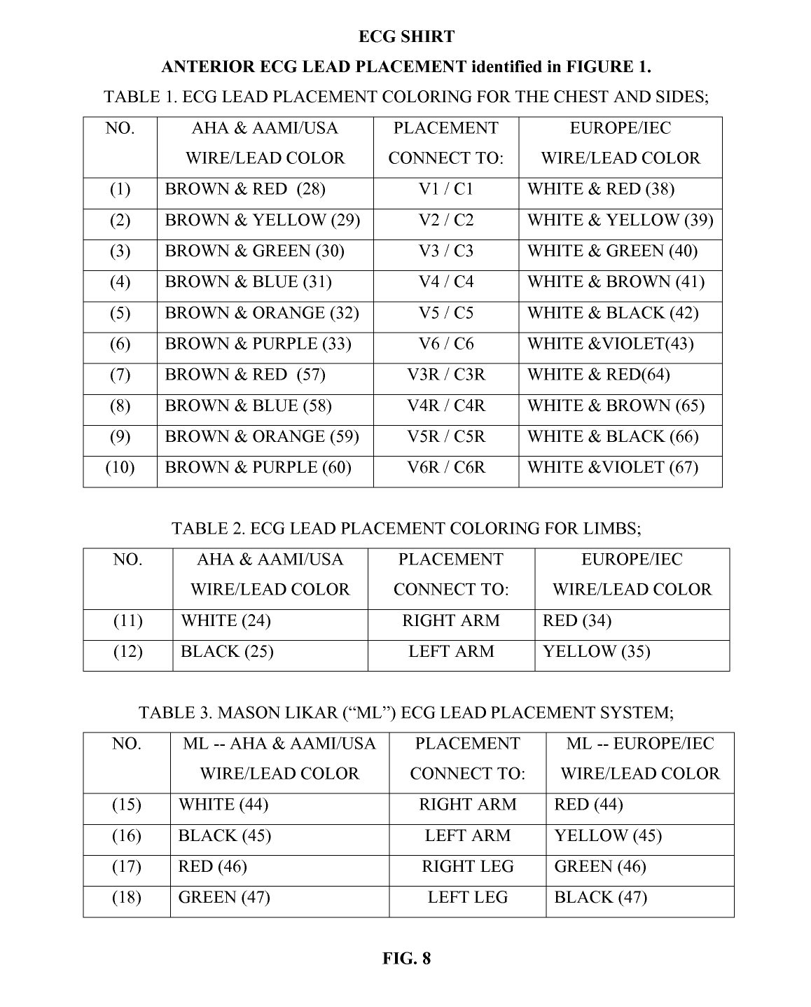

FIG. 9. ECG Shirt Tables 4-5

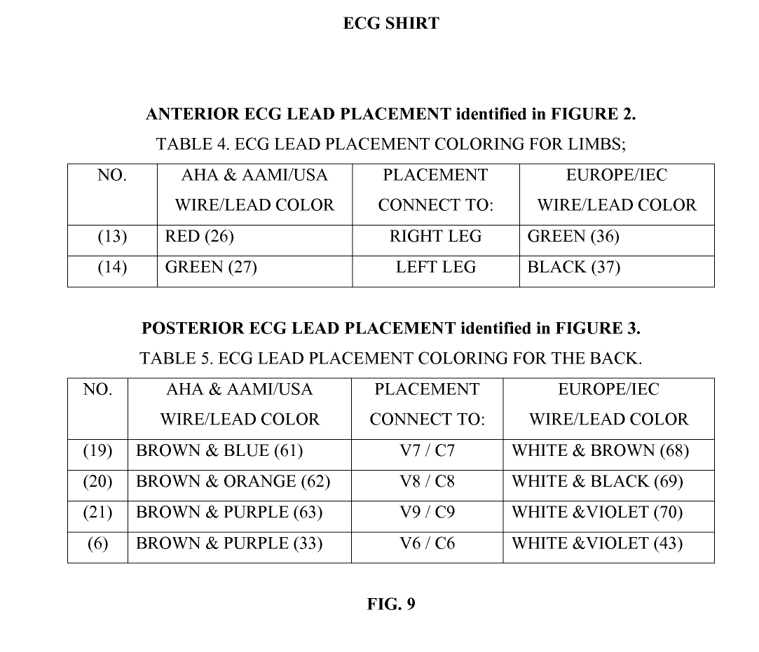

### DETAILED DESCRIPTION OF THE INVENTION

The ECG Shirt is an elastic method and apparatus comprising a garment. The different embodiments of the ECG shirt encompass variations with sleeves; the ECG Shirt and a brassiere with sleeves; the variations without sleeves: a brassiere or a vest without sleeves that take advantage of an elastic fabric such as; polyester, spandex, neoprene, or the like to stretch with the enormous variations of body types and sizes.

The twenty-one apertures correlate with the International Electrotechnical Commission, (IEC); Association for the Advancement of Medical Instrumentation, (AAMI); and American Heart Association (AHA) their recommended ECG electrode lead wire and ECG electrode clips variations on color-coding and labeling schema.

Twenty-one elastic straps are sewn of otherwise attached on the inside of the garment under the corresponding labels, instructions, specific color or plurality of color described by AHA, AAMI, and IEC. The label on the upper side of the strap is visible through the aperture in the ECG Shirt. The elastic strap is not as wide as the aperture to allow for easy elevation for healthcare workers when the will clean the correlated area of skin with alcohol and attach the ECG electrode or an electrode pad with a sticky surface gel on the surface of the skin. When the elastic strap is released and allowed to rest on the ECG electrode pad or electrode it applies constant light pressure and creates a better electrical signal.

Twenty-one thin elastic straps (56) are sewn or fastened externally that fasten the electrode clips to the ECG Shirt. These thin external elastic strips serve two purposes. The first purpose is to harness the ECG electrode wires so that they do not hang loosely away from the body and are easily entangled or accidentally pulled off. The second purpose is for hooking on to the ECG electrode clip. When the thin elastic strap rests over the ECG electrode wire snugly it is able to catch the top portion of the clip so that it will not move away from the ECG Shirt. This additional safety measure eliminates a large portion of risk associated with unexpected accidental motion leading to problematic artifacts in the ECG test record.

Eight smaller elastic straps or apertures (55) are evenly distributed: with two straps or apertures on each of the sleeves; one strap or aperture on each of the shoulders; and one strap or aperture on the left and one strap aperture on the right above the Thoracic Cage near the Clavicle to harness the ECG electrode lead wires. The wires can be easily passed through the aperture or straps to avoid accidental dislocation or movement cause by loose limb wires. This is a very common problem that has resulted in a compromised methodology where the limb leads are place in the Mason Likar system for twelve lead monitoring or for exercise stress testing.

The brassiere is an embodiment of Claim 1 except with a design supporting, cupping, raising, and separating larger fatty breasts common with women. It is also helpful for obese men that may have large breasts. Comprising the embodiment of the ECG Shirt the brassiere uses the straps and apertures correlating to AHA, AAMI, and IEC standards. The brassiere design is very helpful comprising a central panel or stitching directly over the sternum that separates the breasts. The cups support under the breasts and around the breasts to allow for easy ECG lead placement away from the most fatty area of the breasts. The tight fitting section directly under the breast cups similar to a corset covers the abdomen with the snug elastic material down to the hips to provide placement for the Mason Likar System leg lead placement. The brassiere also has the embodiment of Claim 3 where no sleeves are used.

The vest is an embodiment of Claim 1 except for the sleeveless variation. The vest is for people without arms, people who have an arm or arms with restricted mobility, or for people who have other limitations with their arms that may make entrance into a sleeve problematic. The embodiment of Claim 5 provides the necessary limb elements. Due to the large volume of ECG electrode lead placement errors where the lead is place on the wrong arm, there is an additional label on the abdomen at the Left Mid-Clavicular Line spelling “LEFT ARM”; a black arrow pointing left with a label spelling the letters “LA” using white color; a yellow arrow pointing left with a label spelling the letter “L” using black color; an additional label on the abdomen at the Left Mid-Clavicular Line spelling “RIGHT ARM”; a red arrow pointing left with a label spelling the letters “RA” using white color; a white arrow pointing left with a label spelling the letter “R” using black color to emphasize the correct placement.

The four elastic garters (13, 14, 22, 23) with apertures except for the color-coded label on each band that identifies the band as either the (13) right leg, (14) left leg, (22) right arm, or (23) left arm for use with a the embodiments of Claim 2 and the embodiment of Claim 4 where sleeves are problematic.

The aperture of Claim 1 wherein an electrode is able to be correctly and quickly positioned in the desired area on the patient's skin is carefully described by the AHA, AAMI, and IEC. One of the most unique advantages of the ECG Shirt is the ability to provide the majority of ECG electrode placement training so clearly and in front of the person responsible for the patient. The specific anatomical placement areas are; (1) Fourth Intercostal Space at the Right Sternal Border, (2) an electrode is able to be positioned in the Fourth Intercostal Space at the Left Sternal Border, (3) an electrode is able to be positioned mid-way between the Fourth Intercostal Space at the Left Sternal Border and the Fifth Intercostal Space in the Mid-Clavicular Line, (4) an electrode is able to be positioned in the Fifth Intercostal Space in the Left Mid-Clavicular Line, (5) an electrode is able to be positioned in the Left Anterior Axillary Line at the level of the Fifth Intercostal Space, (6) an electrode is able to be positioned in the Left Mid-Axillary Line at the level of the Fifth Intercostal Space, (7) an electrode is able to be positioned mid-way between the Fourth Intercostal Space at the Right Sternal Border and the Fifth Intercostal Space, (8) an electrode is able to be positioned in the Fifth Intercostal Space in the Right Mid-Clavicular Line, (9) an electrode is able to be positioned in the Right Anterior Axillary Line at the level of the Fifth Intercostal Space, (10) an electrode is able to be positioned in the Right Mid-Axillary Line at the level of the Fifth Intercostal Space, (19) an electrode is able to be positioned in the Left Posterior Axillary Line at the level of the Fifth Intercostal Space, (20) an electrode is able to be positioned in the Left Mid-Axillary Line at the level of the Fifth Intercostal Space, (21) an electrode is able to be positioned in the Left Paraspinal Line at the level of the Fifth Intercostal Space, (11) an electrode is able to be positioned on the Right Sleeve at the wrist, (12) an electrode is able to be positioned on the Left Sleeve at the wrist, (15) an electrode is able to be positioned on the Right Deltoid Fossa, Mid-Clavicular Line, (16) Left Deltoid Fossa, Mid-Clavicular Line, (17) Right Anterior Axillary, Mid-Clavicular Line, (18) Left anterior axillary, Mid-Clavicular Line. With the ECG Shirt the course material in their ECG training including advanced cardiovascular recommendations for additional right lead and posterior lead placement is spectacularly easy to understand.

Another simple but major improvement of prior art is the plurality of Labels and Instructions encompassing Claim 1 comprising a letter of the alphabet, a number, a combination of a letter or letters of the alphabet and a number, or spelling. These labels and instructions provide immediate visual aids for the twelve lead, fifteen lead, and eighteen lead ECG placement that is recommended by rarely utilized. The additional training and time necessary for a fifteen lead or eighteen lead placement and the understanding of the importance in properly diagnosing myocardial infarction is not commonly available. The ECG shirt provides the labels according to; IEC, AAMI, and AHA.

The ten color-codes and label schema for the AAMI and AHA is; (11,24) “RA” with the color White, (12,25) “LA” with the color Black, (13,26) “RL” with the color Green, (14,27) “LL” with the color Red, (1,28) “V1” with the color combination of Brown and Red, (2,29) “V2” with the color combination of Brown and Yellow, (3,30) “V3” with the color combination of Brown and Green, (4,31) “V4” with the color combination of Brown and Blue, (5,32) “V5” with the color combination of Brown and Orange, (6,33) “V6” with the color combination of Brown and Purple.

The ten color-codes and label schema for the IEC is; (11,34) “R” with the color Red, (12,35) “L” with the color Yellow, (13,36) “N” with the color Green, (14,37) “F” with the color Black, (1,38) “C1” with the color combination of White and Red, (2,39) “C2” with the color combination of White and Yellow, (3,40) “C3” with the color combination of White and Green, (4,41) “C4” with the color combination of White and Brown, (5,42) “C5” with the color combination of White and Orange, (6,43) “C6” with the color combination of White and Violet,

The four color-codes and label schema for the Mason Likar System of ECG lead placement is; (44) right arm, “RA-ML”, with the colors White and Red, (45) left arm, “LA-ML”, with the colors Black and Yellow, (46) left leg, “LL-ML”, with the color Green, (47) right leg, “RL-ML”, with the colors Red and Black,

The two instruction labels for the fifteen lead placements are placed on the ECG Shirt front and back. The message labels comprising, (48) “Fifteen Lead ECG—V4/C4 cable for V4R position, Fifteen Lead ECG—V5/C5 cable for V8 position, Fifteen Lead ECG—V6 cable for V9 position”;

Three instruction labels for the eighteen lead placements comprising: ANTERIOR/FRONT LABEL; (49) “Eighteen Lead Placement—Move V1/C1 to V4R, Move V2/C2 to V5R″, and Move V3/C3 to V6R″, “Eighteen Lead Placement—Right Ventricle—Move V1/C1 to V3R”, Move V2/C2 to V4R″, and Move V3/C3 to V5R″, and the POSTERIOR/BACK LABEL; (50) “Eighteen Lead Placement—Move V4/C4 to V7”, Move V5/C5 to V8, Move V6 to V9″.

Unique labels for the right leg and the left leg comprise; (52) “ANKLES”; (53) and Two arrows pointing down.

The label that is the most simple straight forward and obvious is designed to eliminate the left are right arm reversals and comprises a very large label on the left sleeve of the ECG Shirt (54) “LEFT ARM” written vertically.

In addition to the standard twelve lead ECG placement patterns the ECG Shirt also provides two eighteen lead placements and one fifteen lead placements that incorporate ECG leads (7,57) V3R/ (7,64) C3R, (8,58) V4R/ (8,65) C4R, (9,59) V5R/(9,66) CSR, (10,60) V6R/ (10,67) C6R, (19,61) V7/(19,68) C7, (20,62) V8/(20,69) C8, and (21,63) V9/(21,70) C9. These additional lead placements are recommended but only used sporadically because of the lack of training available.

I claim:

1. A method and apparatus that defines and secures Electrocardiogram (ECG) lead placement comprising;
1 An elastic method and apparatus comprising; a garment with or without sleeves; an elastic fabric; apertures, straps, and garters, holding firm contact of the ECG leads, garment, and ECG electrodes to the skin; labels, instructions, and color-coding for clear, easy, concise, and clinically diverse ECG electrode lead placement according to international standards;
2 An embodiment of claim 1 except with a design of a brassiere; supporting, cupping, raising, and separating the breasts to facilitate correct ECG lead placement;
3 An embodiment of claim 1 as a vest with an additional label on the abdomen clearly directing placement of the Embodiment of claim 5;
4 An embodiment of claim 2 and an embodiment of claim 3 without sleeves;
5 An embodiment of claim 1 comprising four distinct elastic garters for ECG lead placement of limbs, with apertures identical except for the color and labels, for use with a the embodiments of claim 2 and the embodiment of claim 4;
6 The apertures of claim 1 and the embodiments of claim 2, claim 3, claim 4, and claim 5 wherein an electrode is able to be positioned in the precise anatomical location corresponding with accepted clinical protocols for ECG testing of the twelve lead, fifteen lead, and eighteen lead for testing and monitoring; resting or during exercise;
7 A plurality of Labels and Instructions encompassing claim 1 and the embodiments of claim 2, claim 3, claim 4, claim 5, and claim 6 with bold visual cues and specific instructions according to the highest standards of clinical cardiovascular ECG testing placed in the precise location of ECG lead placement; facilitating and increasing the speed of ECG lead application to a patient' body; and providing additional advanced instruction for vital ECG tests using the right precordial leads and posterior leads;
